(19) United States
(12) Patent Application Publication
(10) Pub. No.: US 2019/0261913 A1
(43) Pub. Date: Aug. 29, 2019

(54) HYPERSPECTRAL IMAGING FOR PASSIVE DETECTION OF COLORECTAL CANCERS

(71) Applicant: The Johns Hopkins University, Baltimore, MD (US)

(72) Inventors: Robert J. Beaulieu, Baltimore, MD (US); Seth D. Goldstein, Baltimore, MD (US); Bashar Safar, Baltimore, MD (US); Amit Banerjee, Baltimore, MD (US); Nita Ahuja, Baltimore, MD (US)

(73) Assignee: The Johns Hopkins University, Baltimore, MD (US)

(21) Appl. No.: 16/342,805

(22) PCT Filed: Oct. 18, 2017
(86) PCT No.: PCT/US2017/057249

§ 371 Date: Apr. 17, 2019

Related U.S. Application Data
(60) Provisional application No. 62/409,769, filed on Oct. 18, 2016.

(51) Int. Cl. A61B 5/00

(52) U.S. Cl. CPC A61B 5/4255; A61B 5/7275; A61B 5/0075; A61B 5/7267

(57) ABSTRACT

A method of detecting colorectal cancers using a hyperspectral sensor system includes receiving a training set of hyperspectral data from colorectal tissue that includes known normal and known cancerous tissue using the hyperspectral sensor system, applying a machine learning algorithm to the training set of hyperspectral data to provide predictor parameters for one of cancerous tissue or non-cancerous tissue, receiving measurement hyperspectral data from colorectal tissue of interest, and using the predictor parameters to classify the colorectal tissue of interest as one of cancerous tissue or non-cancerous tissue. The training set of hyperspectral data and the measurement hyperspectral data include reflection spectra across wavelength bands of light that include at least visible, near infrared and short-wave infrared regions of the electromagnetic spectrum.

### CROSS-REFERENCE TO RELATED APPLICATIONS

This application claims priority benefit from U.S. Provisional Patent Application No. 62/409,769 filed on Oct. 18, 2016, the entire content of which is incorporated herein by reference. All references cited anywhere in this specification, including the Background and Detailed Description sections, are incorporated by reference as if each had been individually incorporated.

### BACKGROUND

### 1. Technical Field

The field of the currently claimed embodiments of this invention relates to hyperspectral sensor systems, methods of detecting colorectal cancers using hyperspectral sensor systems, and computer-executable code for detecting colorectal cancers using hyperspectral sensor systems.

### 2. Discussion of Related Art

Colorectal cancer is the fourth most common cancer in the United States, and is the second leading cause of cancer death, accounting for 49,000 deaths annually.¹ Complete surgical resection, with pathologically negative margins, is the mainstay of therapy in early stage disease. However, obtaining adequate margins can be an inexact science, as current methods for intraoperative decision-making require visual distinction and physical palpation of critical anatomy. Local recurrence, presumably from residual disease at the time of surgery, is responsible for a major amount of the long-term morbidity of the disease.

A method of detecting malignant tissues to complement surgeon visual and haptic perception would immediately be a valuable adjunct in colorectal cancer surgery. Near-infrared fluorescence imaging has been tested for intraoperative use, but is a nonspecific modality that works by simply identifying areas of increased blood flow and requires administration of an exogenous contrast agent.² No targeted fluorophores have been developed to date that would allow recognition of cancer based on intrinsic tissue properties. Thus, technology that explores tissue discrimination based on endogenous spectroscopic properties is quite attractive. Hyperspectral imaging (HSI) is a passive, absorbance-based imaging technique that captures high-resolution spectral signals from across the optical spectrum, including the visible (VIS) through near-infrared (NIR) and shortwave-infrared (SWIR) wavelengths, without the need for ionizing radiation.³

The applications for hyperspectral sensing (HS) for biomedical applications are rapidly becoming areas of increased academic and commercial interest. For medical imaging with hyperspectral sensing, light is directed at a biologic source, at which point it undergoes multiple scattering events due to “inhomogeneity of biologic structures.”³ The hyperspectral detector then captures the reflected light over a characteristic range of wavelengths that can include VIS through the SWIR. The depth of penetration varies from millimeters for visible light to as deep as multiple centimeters for NIR light, which enables capture of data from multiple tissue levels simultaneously.^{12,13} Sensitive and specific medical imaging with hyperspectral sensing relies on the assumption that reflected light contains spectral features that are unique to characteristics of the underlying tissue, including the cellular crowding, amount of blood flow and metabolic activity, and presence of specific physiologic substrates.¹⁴⁻¹⁶

Another barrier to HS investigation of biologic tissues has been a lack of data-analytic techniques. High spectral resolution imaging generates a large amount of data, and robust methods of sample comparison have not been previously determined.

Therefore, there remains a need for improved hyperspectral sensor systems, methods of detecting colorectal cancers using hyperspectral sensor systems, and computer-executable code for detecting colorectal cancers using hyperspectral sensor systems.

### SUMMARY OF THE DISCLOSURE

An aspect of the present disclosure is to provide a method of detecting colorectal cancers using a hyperspectral sensor system. The method include receiving a training set of hyperspectral data from colorectal tissue that includes known normal and known cancerous tissue using the hyperspectral sensor system; and applying a machine learning algorithm to the training set of hyperspectral data to provide predictor parameters for one of cancerous tissue or non-cancerous tissue. The method also includes receiving measurement hyperspectral data from colorectal tissue of interest; and using the predictor parameters to classify the colorectal tissue of interest as one of cancerous tissue or non-cancerous tissue. The training set of hyperspectral data and the measurement hyperspectral data include reflection spectra across wavelength bands of light that include at least visible, near infrared and short-wave infrared regions of the electromagnetic spectrum.

Another aspect of the present disclosure is to provide a hyperspectral sensor system for detecting colorectal cancers. The system includes a hyperspectral illumination source configured to illuminate colorectal tissue, a hyperspectral receiver arranged to receive light from the hyperspectral illumination source after being reflected from the colorectal tissue; and a detection system configured to communicate with the hyperspectral receiver. The detection system is configured to: receive a training set of hyperspectral data from the hyperspectral receiver for colorectal tissue that includes known normal and known cancerous tissue, apply a machine learning algorithm to the training set of hyperspectral data to provide predictor parameters for one of cancerous tissue or non-cancerous tissue, receive measurement hyperspectral data from the hyperspectral receiver for colorectal tissue of interest, and use the predictor parameters to classify the colorectal tissue of interest as one of cancerous tissue or non-cancerous tissue. The hyperspectral illumination source and the hyperspectral receiver illuminate and receive, respectively, light that include at least visible, near infrared and short-wave infrared regions of the electromagnetic spectrum.

A further aspect of the present disclosure is to provide a computer readable medium comprising non-transient computer-executable code for detecting colorectal cancers using a hyperspectral sensor system, the code, when executed by a computer, causes the computer to: receive a training set of hyperspectral data from colorectal tissue that includes known normal and known cancerous tissue using the hyperspectral sensor system; apply a machine learning algorithm to the training set of hyperspectral data to provide predictor parameters for one of cancerous tissue or non-cancerous tissue; receive measurement hyperspectral data from colorectal tissue of interest; and use the predictor parameters to classify the colorectal tissue of interest as one of cancerous tissue or non-cancerous tissue. The training set of hyperspectral data and the measurement hyperspectral data includes reflection spectra across wavelength bands of light that include at least visible, near infrared and short-wave infrared regions of the electromagnetic spectrum.

### BRIEF DESCRIPTION OF THE DRAWINGS

The present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

FIG. 1 is an image of a collected colorectal specimen with white circles indicating sites of extraluminal data collection, according to an embodiment of the present disclosure;

FIG. 2 is a plot of probability of detection of cancer versus probability of false alarm showing a Receiver Operating Characteristics (ROC) curve for tumor detection with extraluminal specimen collection method, according to an embodiment of the present disclosure;

FIG. 3 is a plot of probability of detection of cancer versus probability of false alarm showing a ROC curve for tumor detection with intraluminal specimen collection method, according to another embodiment of the present disclosure; and

FIG. 4 is schematic diagram of a hyperspectral (HS) system, according to an embodiment of the present disclosure.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

In order to facilitate use of HSI as a clinically relevant tool for intraoperative and clinical decision-making according to some embodiments of the current invention, we embarked on a proof-of-concept study with human subjects to understand use of this modality in colorectal cancer, which is an attractive target due the high incidence of the disease and the potential to incorporate novel technology into preoperative endoscopic screening methods and intraoperative techniques. Here, we report the first use of HSI and machine learning classification for both intraluminal and extraluminal tumor detection of colorectal cancer.

The following describes some further concepts of the current invention by way of some particular embodiments. However, the broad concepts of the current invention are not limited to the particular embodiments so described.

### Methods

This study was approved by the Johns Hopkins Hospital Institutional Review Board (IRB). Patients undergoing open or laparoscopic colectomy for resection of colorectal cancer were included in the study. Details of the operative technique, including method of bowel preparation, open versus laparoscopic approach and margins of resection, were at the discretion of the surgeon. In cases in which preoperative biopsy results existed, these data were collected and compared to pathologic results from operative specimen to confirm the presence of malignancy.

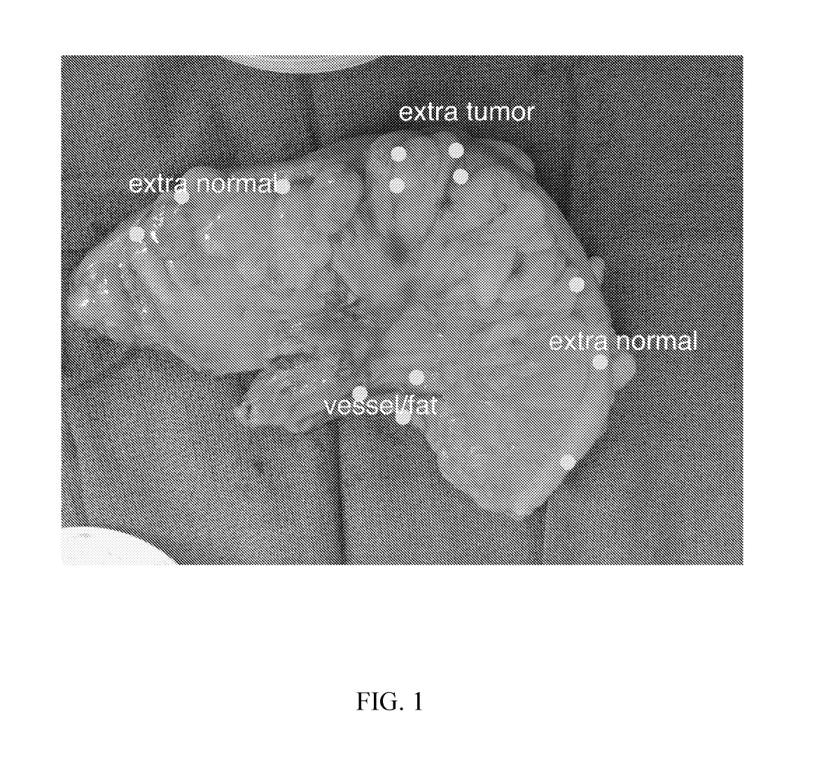

FIG. 1 is an image of a collected colorectal specimen with white circles indicating sites of extraluminal data collection, according to an embodiment of the present disclosure. As shown in the image, normal tissue was collected on either side of the tumor tissue. Multiple samples were taken from each site. Hyperspectral signal collection was conducted on freshly resected colon specimens using point spectroscopy with the Labspec 5000 spectrometer (ASD Inc., Boulder, Colo.). Spectral signals were collected with 1 nm resolution for wavelengths 350 nm to 1800 nm. For each specimen, spectra were obtained from multiple extraluminal points over normal segments of colon at least 10 cm away from tumor, as shown in FIG. 1. Additionally, extraluminal signals were also obtained in the tissue immediately overlying any palpable tumor. Resected specimens were then sent to pathology for gross examination of tumor margins. Specimens were returned to the operating room for intraluminal as well as repeat extraluminal spectral signal acquisition. Both extraluminal and intraluminal signals were obtained of normal colon and tumor, with tumor identification confirmed by the pathologist.

All data processing and analysis was conducting offline with algorithms developed using the Matlab 2016a platform (Mathworks Inc, Natick, Mass.). The first step was to determine if the spectra of normal and tumor tissue samples contain features that are stable within each class and provide separability between normal and tumor tissue. This was done by using Linear Discriminant Analysis (LDA). Given the raw spectral signals with assigning class labels of normal or tumor, LDA transformed the spectra into a single feature that allowed for the greatest degree of separation between the two classes. A feature value threshold was then assigned to generate a classifier for future testing. Following classifier construction, data were cross-validated by using 50% of the data as a “training” set and testing on the remaining 50%. A Monte Carlo (MC) method was used to randomly select data for inclusion into either the training or testing data set over a series of 100 iterations. Following Monte Carlo iterations, results were averaged and used to generate a representative Receiver Operating Characteristics (ROC) curve.

### Results

Fifteen patients (11 male, 4 female), ages 27-90 years, were prospectively enrolled in the study (Table 1). In Table 1 is reported patient demographic data and pathology outcomes among patients with identified cancer in their specimen.

TABLE 1

| PATIENT | AGE | GENDER | TUMOR TYPE | TUMOR SIZE | GRADE | TNM |
|---|---|---|---|---|---|---|
| 1 | 62 | M | Signet Ring | 2.5 cm | High | T2N0M0 |
| | | | Adenocarcinoma | | | |
| 2 | 79 | M | Mucinous | 11 cm | Low | T3N0M0 |
| | | | Adenocarcinoma | | | |
| 3 | 38 | F | Adenocarcinoma | 2 cm | Low | |
| 4 | 61 | M | Mucinous | 6.5 cm | Low | T3N0M0 |
| | | | Adenocarcinoma | | | |
| 5 | 91 | M | Adenocarcinoma | 6.2 cm | Low | T4aN0M0 |
| 6 | 63 | M | Adenocarcinoma | 5.2 cm | Low | T3N0M0 |
| 7 | 66 | M | Adenocarcinoma | 9 cm | Low | T4bN0M0 |
| 8 | 56 | F | Adenocarcinoma | 1.9 cm | Low | T3N0M0 |
| 9 | 67 | M | Adenocarcinoma | 3.7 cm | Low | T2N0M0 |
| 10 | 54 | F | Adenocarcinoma | 6 cm | Low | T3N0M0 |
| 11 | 69 | M | Adenocarcinoma | 5 cm | Low | T3N0M0 |
| 12 | 47 | M | Adenocarcinoma | 4.4 cm | Low | T4N2M0 |

Surgical resection of the affected colon segment was successful in all patients. Fourteen (93%) patients had preoperative colonoscopy demonstrating concerning polyps or colonic mass. A total of three patients were excluded. One patient underwent resection for inflammatory bowel disease (IBD) and polyps. However, postoperative pathologic examination revealed no evidence of adenocarcinoma. In this patient, spectral signals from normal segments of colon were obtained in areas without any evidence of inflammation or polyps and were subsequently included for additional analysis. One patient was excluded after pathologic examination only revealed high grade dysplasia. A third subject was excluded after receiving indocyanine green (ICG) for intraoperative blood flow assessment which interfered with spectral absorbance patterns at wavelengths between 800 and 900 nm. Subsequent analysis of the spectral waveform for this subject demonstrated a significantly altered peak in the expected wavelength for ICG, compared to the remaining subjects. For all included patients, pathologic examination of resected specimens confirmed the diagnosis of adenocarcinoma. Hyperspectral signal acquisition was successfully completed on all resected specimens.

Analysis of extraluminal results before and after pathologic examination did not demonstrate any difference by LDA classification. The average time from resection to reexamination after pathologic evaluation was greater than 30 minutes, suggesting that the hyperspectral features of interest in this study are initially stable despite extirpation and presumptive early ischemia.

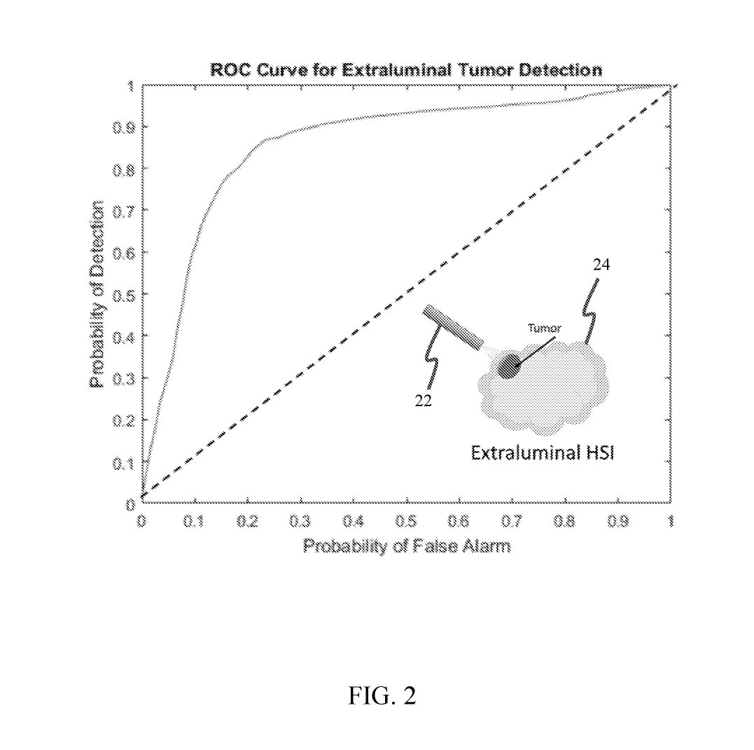

FIG. 2 is a plot of probability of detection of cancer versus probability of false alarm showing a ROC curve for tumor detection with extraluminal specimen collection method, according to an embodiment of the present disclosure. As shown in FIG. 2, the extraluminal collection method corresponds to the detection of tumor in a specimen wherein a spectroscopic probe 22 is outside the specimen 24. The dotted line in the plot represents results similar to chance. The solid line curve corresponds to the ROC curve for extraluminal tumor detection (extraluminal HSI). As shown in FIG. 2, for specimens imaged extraluminally, a series of thresholds for cancer detection were used to create the ROC curve (solid line), which demonstrated detection rates significantly greater than chance (dotted line). When a cutoff threshold for 5% false positive rate was set, the rate of cancer detection was 30.73%. When this false positive rate was increased to 10%, the tumor detection rate increased to 61.68%.

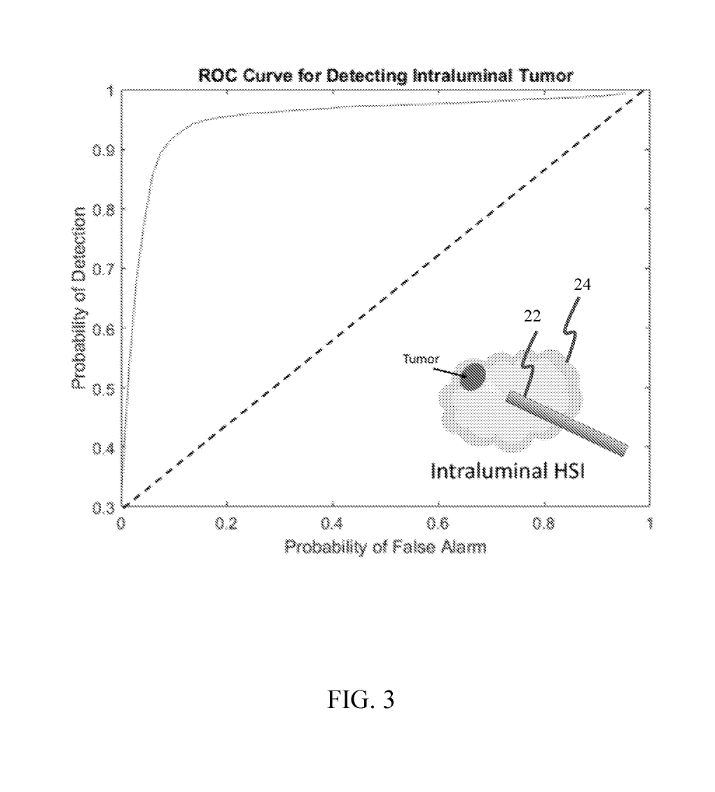

FIG. 3 is a plot of probability of detection of cancer versus probability of false alarm showing a ROC curve for tumor detection with intraluminal specimen collection method, according to another embodiment of the present disclosure. As shown in FIG. 3, the intraluminal collection method corresponds to the detection of tumor in a specimen wherein a spectroscopic probe 22 is inside the specimen 24. The dotted line in the plot represents results similar to chance. The solid line curve corresponds to the ROC curve for intraluminal tumor detection (intraluminal HSI). As shown in FIG. 3, for intraluminal specimens, the averaged ROC curve (solid line) demonstrated higher overall detection rates at every false positive threshold compared to the averaged ROC curve (solid line) of the extraluminal imaging method (the solid curve in FIG. 2). The rate of detection with the 5% false positive threshold was 80.76% and increased to 91.97% when the false positive rate was increased to 10%.

To optimize classification of tumors by intraluminal detection we explored an additional classification method in a post-hoc manner for detection rate analysis. Support Vector Machines (SVM) classification method allows for non-linear borders between groups of data. When this method was applied to the intraluminal data, again using two classes (normal and tumor), the detection rate was 86% with 5% false positive and yy5 with 10% false positive rate.

### Discussion

Local control following colectomy for colorectal cancer is heavily dependent on obtaining pathologically negative margins, a so-called R0 resection, at the time of operation.¹⁰ Currently, surgeons rely on visual inspection and manual palpation to guide the extent of resection of colorectal surgery, and the latter is absent when a laparoscopic approach is employed. Technology that discriminates tissue based on intrinsic differences in cell biology and vascular supply could be a valuable adjunct in the operating room.¹¹ The first step toward that goal is the development of an approach that reliably detects the presence or absence of cancer in a certain spatial location. Hyperspectral sensing has promise in that regard, but has never successfully been used to discriminate between normal bowel and bowel containing malignant tumor. Here, we have reported the first successful use of HSI in combination with machine learning data processing algorithms to successfully characterize the presence or absence of cancer in a surgical specimen via either intraluminal or extraluminal methods, with sensitivity and specificity of as high as 92% and 90%, respectively.

HSI has been previously applied to astronomy, vegetation analysis and target detection,⁴⁻⁶ but has only recently been applied to biologic tissues and the detection of malignancy. An example of HSI in human xenograft murine models to detect residual tumor following resection has been reported.⁷ However, inherent differences between spectral properties of mouse and human tissues have not been explored, which may preclude generalization of the results to resection of tumor from histologically similar surrounding tissue as in human cancers In a human model of resected gastric cancer specimens, tumor designation based on hyperspectral sensing demonstrated a high correlation with subsequent pathologic determination of malignancy.⁸ In a small group of patients with colorectal cancer, the ability to detect tumor compared to normal tissue when imaged intraluminally was attempted.⁹ However, these studies are limited to proof of principle in small sample size and only used limited spectra.

We found that analysis of extraluminal results before and after pathologic examination did not demonstrate any difference by LDA classification. The average time from resection to reexamination after pathologic evaluation was greater than 30 minutes, suggesting that the hyperspectral features of interest in this study are initially stable despite extirpation and presumptive early ischemia. This is in stark contrast to many of the previously reported optical enhancement models that focus on near infrared signaling and thus are primarily affected by oxygen tension rather than intrinsic tissue properties.

In this work we have used machine-learning algorithms to classify tissues empirically on the basis of their spectral features, without granular knowledge of the nature of the differences between them. Characterization of the spectral patterns generated by normal and diseased tissue will allow for the potential of a sensitive, non-invasive diagnostic imaging technique with no ionizing radiation exposure. Encouraging initial investigations of tissue optics have demonstrated multiple intracellular and extracellular endogenous fluorophores, whose combination in each tissue can help to create a unique spectral absorption/emission pattern.³ From the perspective of oncologic imaging, it is beneficial that some of these tissue fluorophores are involved in process of cellular metabolism (nicotinamide adenine dinucleotide [NADH] and flavin adenine dinucleotide [FAD]) and some are involved in extracellular support (collagen and elastin). Spectral signals with altered patterns of these fluorophores may help identify highly metabolically active cells and those with altered extracellular support matrices. Additionally, spectral signals are affected by alterations in nuclear/cytoplasmic ratio. This combination of altered metabolic activity and change in cellular structure represent key steps along the pathways of malignant degeneration of normal tissue to subsequent cancer.^{3,8}

Due to the presence of these endogenous fluorophores and alteration of the spectral signal with changes in nuclear-to-cytoplasmic ratio, hyperspectral sensing has been used for imaging of cancer in the laboratory setting. In animal models, hyperspectral sensing has been demonstrated high sensitivity and specificity of prostate cancer, melanoma, head and neck cancers and even some non-invasive lesions.^{7,21-24} However, in many of these models, detection accuracy may be bolstered by the difference in human cancer cells in the setting of surrounding normal murine tissue. In human studies, Schol et al have used hyperspectral sensing to characterize normal tissue types, including ureter, fat and vessels, encountered during laparoscopic and open surgery.^{9,25} However, even in this setting, colon cancer detection was measured by a comparison of spectra generated with colon cancer compared to all other healthy tissue, including ureter, fat and vessels, instead of limiting its comparison to normal colon tissue.⁹ Additionally, the data collected were on a limited number of patients (n=6) and performed through intraluminal measurements only. Therefore, we sought to further report a novel and realistic assessment of the usability of hyperspectral sensing in detection of colorectal cancer.

In the data presented herein, hyperspectral sensing demonstrated a high rate of cancer detection when imaged both intraluminally and extraluminally. The lower classification accuracy with extraluminal measurements is likely due to the fact that these samples represented a combination of spectral features from both tumor and the normal colon through which light must first pass before reaching tumor. We believe these results have significant implications for potential technologies to benefit surgical resection of colorectal cancer. Indeed, extraluminal assessment of tumor could be incorporated into an augmented reality format, with an overlay of the malignant potential of each tissue in the surgeon's operative view. The feasibility of augmented reality technologies in the operating room is an area of current exploration.²⁶ Augmented reality technologies, such as Google Glass or Microsoft Hololens, are widely currently available, and the addition of hyperspectral sensing information could allow for improved identification of colorectal cancers, identification of malignancy in peritoneal implants, and detection of synchronous, undiagnosed malignancy in other areas of the bowel.

Intraluminal imaging of potential cancer could significantly aid with the endoscopic detection of colorectal cancer. The incorporation of spectral information onto the video output during a colonoscopy could potentially help to reduce the 2-6% rate of missed cancer following colonoscopy and would also be useful for screening of high risk patient populations such as those with inflammatory bowel disease (IBD) in which the risk of missed malignancy increases to 15%.^{27,28} A post-hoc analysis of the patients with inflammatory bowel disease (n=2) in this study demonstrated correct classification to the non-tumor group in every instance, though a larger study would be needed to validate these findings in this population. Spectral biomarkers have even played a role assessing premalignant colon lesions under the microscope, with promising results.²⁹ We believe our results, in conjunction with previous results reporting high accuracy rates in intraluminal tumors in resected specimens, suggest the useful role of hyperspectral sensing in this setting.⁹

Within our study, the tumor size is typically greater than 3 cm and all lesions are determined to be adenocarcinoma. The possible use of hyperspectral sensing in identifying adenomas or determining their malignant potential although not assessed herein can be investigated using the present HSI technique and method. Additionally, although the largest series reported to date, this study still samples a relatively small patient population. It may be worthwhile to extend this study to a larger sample population set to determine the generalizability of spectral shifts of tumors in the larger population.

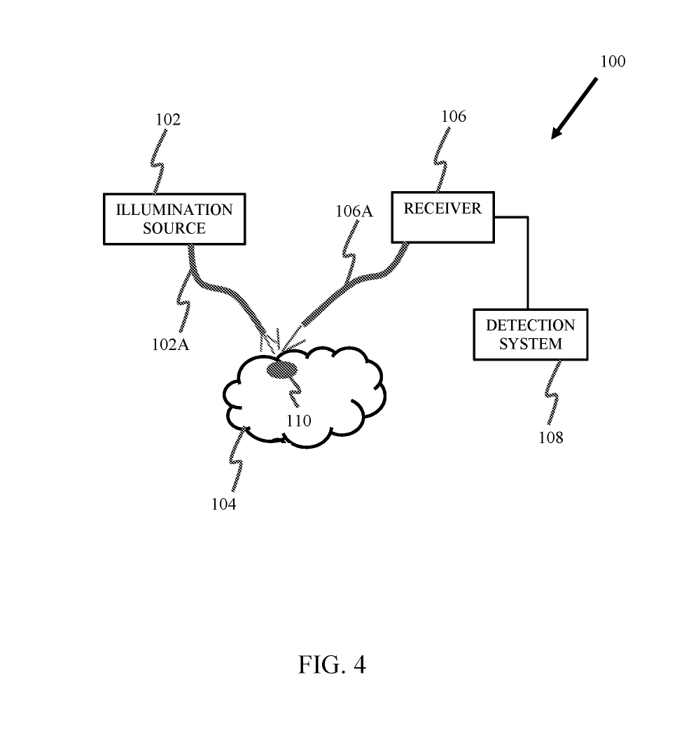

As it can be further appreciated from the above paragraphs, there is provided a hyperspectral sensor (HS) system 100 for detecting colorectal cancers. FIG. 4 is schematic diagram of the HS system 100, according to an embodiment of the present disclosure. As shown in FIG. 4, the HS system 100 includes a hyperspectral illumination source 102 configured to illuminate colorectal tissue 104. For example, the illumination source 102 can illuminate the colorectal tissue 104 using a light guide (e.g., optical fiber) 102A. The HS system 100 also includes a hyperspectral receiver 106 arranged to receive light from the hyperspectral illumination source 102 after being reflected from the colorectal tissue 104. For example, the receiver (e.g., probe) 106 can include a light guide (e.g., an optical fiber) 106A to receive the reflected light via the light guide 106A. The HS system 100 also includes a detection system 108 configured to communicate with the hyperspectral receiver 106. The detection system 108 is configured to:
(a) receive a training set of hyperspectral data from the hyperspectral receiver 106 for colorectal tissue that includes known normal and known cancerous tissue;
(b) apply a machine learning algorithm to the training set of hyperspectral data to provide predictor parameters for one of cancerous tissue or non-cancerous tissue;
(c) receive measurement hyperspectral data from the hyperspectral receiver 106 for colorectal tissue of interest 110, and
(d) use the predictor parameters to classify the colorectal tissue of interest 110 as one of cancerous tissue or non-cancerous tissue.

The hyperspectral illumination source 102 and the hyperspectral receiver 106 illuminate and receive, respectively, light that include at least visible, near infrared and short-wave infrared regions of the electromagnetic spectrum.

In an embodiment, the training set of hyperspectral data and the measurement hyperspectral data are from extraluminal colorectal tissue. In another embodiment, the training set of hyperspectral data and the measurement hyperspectral data are from intraluminal colorectal tissue.

In an embodiment, the hyperspectral illumination source 102 and the hyperspectral receiver 106 illuminate and receive, respectively, across a wavelength range from about 350 nm to 1800 nm. In an embodiment, the hyperspectral illumination source 102 and the hyperspectral receiver 106 illuminate and receive, respectively, across a wavelength range from about 350 nm to 1800 nm with a resolution of at least 1 nm.

In an embodiment, the machine learning algorithm uses a Linear Discriminant Analysis method. For example, the machine learning algorithm uses a Linear Discriminant Analysis method to extract features and uses classifiers such as support vector machines (SVMs) to classify the signal.

In an embodiment, the detection system 108 may include a computer readable medium comprising non-transient computer-executable code for detecting colorectal cancers using a hyperspectral sensor system, the code, when executed by a computer, causes the computer to:
(a) receive a training set of hyperspectral data from colorectal tissue that includes known normal and known cancerous tissue using the hyperspectral sensor system;
(b) apply a machine learning algorithm to the training set of hyperspectral data to provide predictor parameters for one of cancerous tissue or non-cancerous tissue;
(c) receive measurement hyperspectral data from colorectal tissue of interest; and
(d) use the predictor parameters to classify the colorectal tissue of interest 110 as one of cancerous tissue or non-cancerous tissue,

The training set of hyperspectral data and the measurement hyperspectral data comprise reflection spectra across wavelength bands of light that include at least visible, near infrared and short-wave infrared regions of the electromagnetic spectrum.

As it can also be appreciated from the above paragraphs, there is further provided a method of detecting colorectal cancers using a hyperspectral sensor system 100, according to an embodiment of the present disclosure. The method includes receiving a training set of hyperspectral data from colorectal tissue that includes known normal and known cancerous tissue using the hyperspectral sensor system 100. The method also includes applying a machine learning algorithm to the training set of hyperspectral data to provide predictor parameters for one of cancerous tissue or non-cancerous tissue. The method further includes receiving measurement hyperspectral data from colorectal tissue of interest 110; and using the predictor parameters to classify the colorectal tissue of interest 110 as one of cancerous tissue or non-cancerous tissue. The training set of hyperspectral data and the measurement hyperspectral data comprise reflection spectra across wavelength bands of light that include at least visible, near infrared and short-wave infrared regions of the electromagnetic spectrum.

The term “computer” or “computer system” is used herein to encompass any data processing system or processing unit or units. The computer system may include one or more processors or processing units. The computer system can also be a distributed computing system. The computer system may include, for example, a desktop computer, a laptop computer, a handheld computing device such as a PDA, a tablet, a smartphone, etc. A computer program product or products may be run on the computer system to accomplish the functions or operations described in the above paragraphs. The computer program product includes a computer readable medium or storage medium or media having instructions stored thereon used to program the computer system to perform the functions or operations described above. Examples of suitable storage medium or media include any type of disk including floppy disks, optical disks, DVDs, CD ROMs, magnetic optical disks, RAMs, EPROMs, EEPROMs, magnetic or optical cards, hard disk, flash card (e.g., a USB flash card), PCMCIA memory card, smart card, or other media. Alternatively, a portion or the whole computer program product can be downloaded from a remote computer or server via a network such as the internet, an ATM network, a wide area network (WAN) or a local area network.

Stored on one or more of the computer readable media, the program may include software for controlling both the hardware of a general purpose or specialized computer system or processor. The software also enables the computer system or processor to interact with a user via output devices such as a graphical user interface, head mounted display (HMD), etc. The software may also include, but is not limited to, device drivers, operating systems and user applications. Alternatively, instead or in addition to implementing the methods described above as computer program product(s) (e.g., as software products) embodied in a computer, the method described above can be implemented as hardware in which for example an application specific integrated circuit (ASIC) or graphics processing unit or units (GPU) can be designed to implement the method or methods, functions or operations of the present disclosure.

### Conclusion

Hyperspectral sensing can reliably detect tumors using both intraluminal and extraluminal measurements. The technology is non-ionizing and does not require the use of contrast agents to achieve accurate colorectal cancer detection.

### REFERENCES

1. Ryerson, A. B. et al. Annual Report to the Nation on the Status of Cancer, 1975-2012, featuring the increasing incidence of liver cancer. Cancer 122, 1312-37 (2016).
2. Frangioni, J. V. In vivo near-infrared fluorescence imaging. Curr. Opin. Chem. Biol. 7, 626-634 (2003).
3. Lu, G. & Fei, B. Medical hyperspectral imaging: a review. J. Biomed. Opt. 19, 10901 (2014).
4. Hege, E. K., O'Connell, D., Johnson, W., Basty, S. & Dereniak, E. L. Hyperspectral imaging for astronomy and space surveillance. in Optical Science and Technology, SPIE's 48th Annual Meeting (eds. Shen, S. S. & Lewis, P. E.) 380-391 (International Society for Optics and Photonics, 2004). doi:10.1117/12.506426
5. Ye, X., Sakai, K., Okamoto, H. & Garciano, L. O. A ground-based hyperspectral imaging system for characterizing vegetation spectral features. Comput. Electron. Agric. 63, 13-21 (2008).
6. Kolodner, M. A. Automated target detection system for hyperspectral imaging sensors. Appl. Opt. 47, F61 (2008).
7. Panasyuk, S. V et al. Medical hyperspectral imaging to facilitate residual tumor identification during surgery. Cancer Biol. Ther. 6, 439-46 (2007).
8. Akbari, H., Uto, K., Kosugi, Y., Kojima, K. & Tanaka, N. Cancer detection using infrared hyperspectral imaging. Cancer Sci. 102, 852-7 (2011).
9. Schols, R. M., Dunias, P., Wieringa, F. P. & Stassen, L. P. S. Multispectral characterization of tissues encountered during laparoscopic colorectal surgery. Med. Eng. Phys. 35, 1044-50 (2013).
10. Atsushi, I. et al. Long-term outcomes and prognostic factors of patients with obstructive colorectal cancer: A multicenter retrospective cohort study. World J. Gastroenterol. 22, 5237-45 (2016).
11. Best, S. L. Hyperspectral imaging in the operating room: what a surgeon wants. 8254, 825403 (2012).
12. Salzer, R., Steiner, G., Mantsch, H. H., Mansfield, J. & Lewis, E. N. Infrared and Raman imaging of biological and biomimetic samples. Fresenius. J. Anal. Chem. 366, 712-6
13. Nouvong, A. et al. Evaluation of diabetic foot ulcer healing with hyperspectral imaging of oxyhemoglobin and deoxyhemoglobin. Diabetes Care 32, 2056-61 (2009).
14. Costas, B., Christos, P. & George, E. in Handbook of Biomedical Optics 131-164 (CRC Press, 2011).
15. Patterson, M. S., Wilson, B. C. & Wyman, D. R. The propogation of optical radiation in tissue I. Models of radiation transport and their application. Lasers Med. Sci. 6, 155-168 (1991).
16. Joel, M. & Tuan, V.-D. in Biomedical Photonics Handbook 1-76 (CRC Press, 2003).
17. Khaodhiar, L. et al. The use of medical hyperspectral technology to evaluate microcirculatory changes in diabetic foot ulcers and to predict clinical outcomes. Diabetes Care 30, 903-10 (2007).
18. Cancio, L. C. et al. Hyperspectral imaging: a new approach to the diagnosis of hemorrhagic shock. J. Trauma 60, 1087-95 (2006).
19. Akbari, H., Kosugi, Y., Kojima, K. & Tanaka, N. Detection and analysis of the intestinal ischemia using visible and invisible hyperspectral imaging. IEEE Trans. Biomed. Eng. 57, 2011-7 (2010).
20. Zuzak, K. J. et al. Intraoperative bile duct visualization using near-infrared hyperspectral video imaging. Am. J. Surg. 195, 491-7 (2008).
21. Akbari, H. et al. Hyperspectral imaging and quantitative analysis for prostate cancer detection. J. Biomed. Opt. 17, 76005 (2012).
22. Lu, G., Halig, L., Wang, D., Chen, Z. G. & Fei, B. Spectral-Spatial Classification

Using Tensor Modeling for Cancer Detection with Hyperspectral Imaging. Proc. SPIE—the Int. Soc. Opt. Eng. 9034, 903413 (2014).
23. Gaudi, S. et al. Hyperspectral imaging of melanocytic lesions. Am. J. Dermatopathol. 36, 131-6 (2014).
24. Leachman, S. A. et al. Methods of Melanoma Detection. Cancer Treat. Res. 167, 51-105 (2016).
25. Schols, R. M. et al. Automated Spectroscopic Tissue Classification in Colorectal Surgery. Surg. Innov. 22, 557-67 (2015).
26. KleinJan, G. H. et al. Towards (hybrid) navigation of a fluorescence camera in an open surgery setting. J. Nucl. Med. (2016). doi:10.2967/jnumed.115.171645
27. Wang, Y. R., Cangemi, J. R., Loftus, E. V & Picco, M. F. Rate of early/missed colorectal cancers after colonoscopy in older patients with or without inflammatory bowel disease in the United States. Am. J. Gastroenterol. 108, 444-9 (2013).
28. Bressler, B. et al. Rates of new or missed colorectal cancers after colonoscopy and their risk factors: a population-based analysis. Gastroenterology 132, 96-102 (2007).
29. Roy, H. K. et al. Spectral biomarkers for chemoprevention of colonic neoplasia: a placebo-controlled double-blinded trial with aspirin. Gut (2015). doi:10.1136/gutjnl-2015-309996

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. A method of detecting colorectal cancers using a hyperspectral sensor system, comprising:
receiving a training set of hyperspectral data from colorectal tissue that includes known normal and known cancerous tissue using said hyperspectral sensor system;
applying a machine learning algorithm to said training set of hyperspectral data to provide predictor parameters for one of cancerous tissue or non-cancerous tissue;
receiving measurement hyperspectral data from colorectal tissue of interest; and
using said predictor parameters to classify said colorectal tissue of interest as one of cancerous tissue or non-cancerous tissue,
wherein said training set of hyperspectral data and said measurement hyperspectral data comprise reflection spectra across wavelength bands of light that include at least visible, near infrared and short-wave infrared regions of the electromagnetic spectrum.

2. The method of claim 1, wherein said training set of hyperspectral data and said measurement hyperspectral data are from extraluminal colorectal tissue.

3. The method of claim 1, wherein said training set of hyperspectral data and said measurement hyperspectral data are from intraluminal colorectal tissue.

4. The method of claim 1, wherein said training set of hyperspectral data and said measurement hyperspectral data comprise reflection spectra across wavelength range from about 350 nm to 1800 nm.

5. The method of claim 4, wherein said training set of hyperspectral data and said measurement hyperspectral data comprise reflection spectra across having a resolution of at least 1 nm across the wavelength range from about 350 nm to 1800 nm.

6. The method of claim 1, wherein said machine learning algorithm uses a Linear Discriminant Analysis method.

7. The method of claim 1, wherein said machine learning algorithm uses a Linear Discriminant Analysis method to extract features and uses classifiers such as support vector machines (SVMs) to classify the signal.

8. A hyperspectral sensor system for detecting colorectal cancers, comprising:
a hyperspectral illumination source configured to illuminate colorectal tissue;
a hyperspectral receiver arranged to receive light from said hyperspectral illumination source after being reflected from said colorectal tissue; and
a detection system configured to communicate with said hyperspectral receiver,
wherein said detection system is configured to:
receive a training set of hyperspectral data from said hyperspectral receiver for colorectal tissue that includes known normal and known cancerous tissue,
apply a machine learning algorithm to said training set of hyperspectral data to provide predictor parameters for one of cancerous tissue or non-cancerous tissue,
receive measurement hyperspectral data from said hyperspectral receiver for colorectal tissue of interest, and
use said predictor parameters to classify said colorectal tissue of interest as one of cancerous tissue or non-cancerous tissue,
wherein said hyperspectral illumination source and said hyperspectral receiver illuminate and receive, respectively, light that include at least visible, near infrared and short-wave infrared regions of the electromagnetic spectrum.

9. The hyperspectral sensor system of claim 8, wherein said training set of hyperspectral data and said measurement hyperspectral data are from extraluminal colorectal tissue.

10. The hyperspectral sensor system of claim 8, wherein said training set of hyperspectral data and said measurement hyperspectral data are from intraluminal colorectal tissue.

11. The hyperspectral sensor system of claim 8, wherein said hyperspectral illumination source and said hyperspectral receiver illuminate and receive, respectively, across a wavelength range from about 350 nm to 1800 nm.

12. The hyperspectral sensor system of claim 11, wherein said hyperspectral illumination source and said hyperspectral receiver illuminate and receive, respectively, across a wavelength range from about 350 nm to 1800 nm with a resolution of at least 1 nm.

13. The hyperspectral sensor of claim 8, wherein said machine learning algorithm uses a Linear Discriminant Analysis method.

14. The hyperspectral sensor of claim 8, wherein said machine learning algorithm uses a Linear Discriminant Analysis method to extract features and uses classifiers such as support vector machines (SVMs) to classify the signal.

15. A computer readable medium comprising non-transient computer-executable code for detecting colorectal cancers using a hyperspectral sensor system, said code, when executed by a computer, causes the computer to:
receive a training set of hyperspectral data from colorectal tissue that includes known normal and known cancerous tissue using said hyperspectral sensor system;
apply a machine learning algorithm to said training set of hyperspectral data to provide predictor parameters for one of cancerous tissue or non-cancerous tissue;
receive measurement hyperspectral data from colorectal tissue of interest; and
use said predictor parameters to classify said colorectal tissue of interest as one of cancerous tissue or non-cancerous tissue,
wherein said training set of hyperspectral data and said measurement hyperspectral data comprise reflection spectra across wavelength bands of light that include at least visible, near infrared and short-wave infrared regions of the electromagnetic spectrum.

16. The computer readable medium of claim 15, wherein said machine learning algorithm uses a Linear Discriminant Analysis method.

17. The computer readable medium of claim 15, wherein said machine learning algorithm uses a Linear Discriminant Analysis method to extract features and uses classifiers such as support vector machines (SVMs) to classify the signal.
